(19) United States
(12) Patent Application Publication
(10) Pub. No.: US 2012/0283573 A1
(43) Pub. Date: Nov. 8, 2012

(54) DETECTION DEVICE FOR CHINESE MEDICAL INSPECTION DIAGNOSIS

(75) Inventors: Aimin Gong, Shanghai (CN); Dan Di, Shanghai (CN); Peng Qian, Shanghai (CN); Yue Pan, Shanghai (CN); Yiqin Wang, Shanghai (CN); Fufeng Li, Shanghai (CN)

(73) Assignee: Shanghai Daosheng Medical Technology Co., Ltd., Shanghai (CN)

(21) Appl. No.: 13/143,886

(22) PCT Filed: May 10, 2010
(86) PCT No.: PCT/CN2010/000662

§ 371 Date: Sep. 7, 2011

(30) Foreign Application Priority Data
Sep. 1, 2009 (CN) 200910057863.1

(51) Int. Cl. A61B 6/00

(52) U.S. Cl. 600/476

(57) ABSTRACT

The present invention is to provide a monitoring device for inspection diagnosis in traditional Chinese medicine, comprising: a housing, a light source and a shooting structure, the said light source and shooting structure are positioned in the housing, a photoconductive component is positioned in the said housing, the said photoconductive component comprising at least one photoconductive layer, the said light source is positioned inside the photoconductive layer or on the side cross section of the photoconductive layer, with the diffuse reflection of the photoconductive component the said light source forms a area light source. In the present invention, as the photoconductive component is adopted in light transmission, the point light is converted to area light so that a soft, stable and uniform illumination environment similar to the natural light comes into being in the monitoring device.

### FIELD OF INVENTION

The invention is related to a monitoring device used in traditional Chinese medicine, specifically a monitoring device for the inspection diagnosis of traditional Chinese medicine.

### BACKGROUND

The primary diagnostic information sources in traditional Chinese are the inspection, auscultation and olfaction, inquiry, and feeling the pulse, namely the four diagnostic methods. Wherein, the inspection consists essentially of tongue diagnosis and face diagnosis. The information collection in the traditional inspection diagnosis relies largely on subjectively qualitative observation and clinical experience of the doctor, thus leads to the unfavorable factors of subjective dependence and poor reproducibility, and is very inconvenient to keep a record of the disease.

In order to actualize the objectivity, quantification and standardization of the traditional Chinese inspection diagnosis, the photographic sampling device is adopted to pick-up the images of inspection information so as to judge the condition of the human body according to the color, shape, texture and other characteristics information of the image. The traditional way of collecting inspection diagnostic information is by directly open shot, but it is intended to be disturbed by the light outside which will lead to the color distortion of the images collected, for example the color of the images shall be changed according to the difference of the illumination or light color; later it is was proposed to collect the inspection diagnosis information by using a closed collecting black box, such as the Chinese utility model patent ZL200720172978.1 (Noticed on Oct. 29, 2008) discloses a tongue image collecting black box, which ensures the quality of the color of the tongue image with the confined space and the uniform positioning.

Similar collecting black box (as shown in FIG. 1 and FIG. 2) usually comprises structurally a housing 1, a light source 2 and the shooting structure 3, but the light source 2 is typically directly located in the housing 1 in order to provide the necessary illumination to the shooting structure 3 in the form of point light, thus the drawback lies in the uneven illumination conditions, and with the impact of the light source, there is a large deviation between the light and the natural light, therefore the image captured shall be not consistent with the direct inspection diagnostic information of the doctor, it may result in the misdiagnosis.

### SUMMARY

The technical problem that the present invention needs to solve is to provide a monitoring device for inspection diagnosis in traditional Chinese medicine, which can provide a soft, stable and uniform illumination environment similar to the natural light for shooting.

To solve the above technical problems, the present invention provides a monitoring device for inspection diagnosis in traditional Chinese medicine, which comprising a housing, a light source and a shooting structure, the said light source and shooting structure are positioned in the said housing, a photoconductive component is positioned in the said housing, the said photoconductive component comprising at least one photoconductive layer , the said light source is positioned inside the photoconductive layer or on the side cross section of the photoconductive layer, with the diffuse reflection of the photoconductive component the said light source forms a area light source.

In the present invention, as the photoconductive component is adopted in light transmission, the point lights is converted to area light so that a soft , stable and uniform illumination environment similar to the natural light comes into being in the monitoring device.

### BRIEF DESCRIPTION OF THE DRAWINGS

Preferred embodiments of the present invention will now be more particularly described with reference to the accompanying drawings, wherein:

FIG. 1 is the external structure view of current monitoring device for inspection diagnosis in traditional Chinese medicine;

FIG. 2 is the internal structure view of current monitoring unit for inspection diagnosis in traditional Chinese medicine;

FIG. 3 is the schematic diagram of the photoconductive component in the invention;

FIG. 4 is the structure schematic diagram of shooting environment in the invention;

FIG. 5 is the structure schematic diagram of photoconductive component with LED fixed in the invention;

FIG. 6 is the structure schematic diagram of the LED positioning bar in the invention;

FIG. 7 is the structure schematic diagram of photoconductive component with shooting window frame fixed in the invention;

FIG. 8 is the structure schematic diagram of the shooting window frame in the invention.

### DETAILED DESCRIPTION OF PREFERRED EMBODIMENTS

Based on the shortcomings of the currently inspection diagnosis shooting device, the shooting box of the present invention comprises the traditional housing, light source and the shooting structure as well as the photoconductive component 5. The photoconductive component 5 includes at least one photoconductive layer, preferably, the photoconductive component 5 includes reflective layer, uniform light layer and photoconductive layer from the outside to the inside (besides, if only the reflective layer and the photoconductive layer or the uniform light layer and the photoconductive layer are adopted, which belong to the modified embodiments of the present invention all, the illumination environment is not as effective as when all the three layers are adopted). Two ways can be used in setting the light source, one is to embed directly the light source into the photoconductive layer, and the other is to fix the light source on the side cross section of the photoconductive layer. The light from light source is transmitted in the photoconductive layer by diffuse reflection, thus transfers the point light source and line light source into area light source. The uniform light layer is used to soften the light and make it more similar to the natural light. The reflective layer is used to prevent the internal light from the light loss by the action of reflection, as well as to reflect the external light to avoid the influence to the internal environment. Thus, through the diffuse reflection of the photoconductive component, the light source eventually forms a uniform area light source.

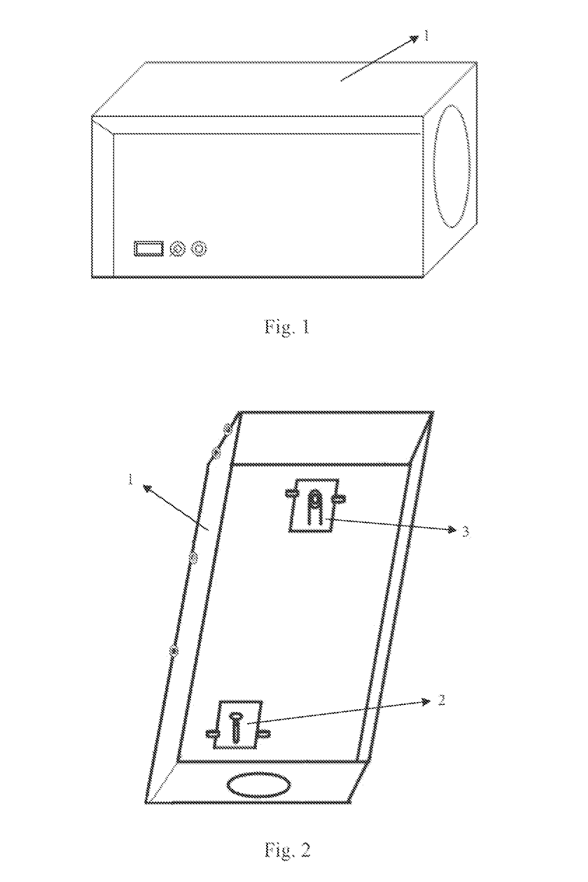

As one simple embodiment of the invention, photoconductive component with the shape of flat plate, polygon (e.g. square, pentagon, hexagon) or curved surface is positioned inside the currently shooting black box (as shown in FIG. 1 and FIG. 2), with which the area light source is formed in the shooting box, and during the shooting procedure, when the light source is turned on, as the shooting environment a stable illuminated environment comes into being. The interior of the photoconductive component is a closed shooting environment, the shooting structure is positioned inside the shooting environment, and the lens is focused on the front panel or the person being inspected.

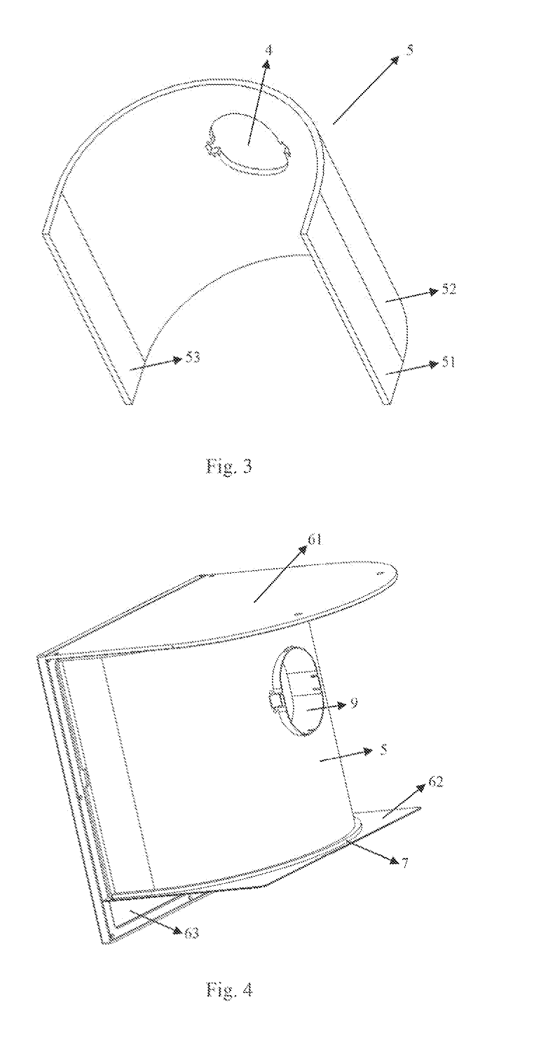

The photoconductive layer of the present invention can be formed with one photoconductive plate or a mosaic composed of at least one photoconductive plate, as shown in FIG. 3, the first photoconductive plate 51, the second photoconductive plate 52 and the third photoconductive plate 53 are joint together and form the photoconductive layer. A white diffuse reflection paste layer (e.g. white diffuse reflection paper), white diffuse reflection plating or coating (e.g. white painting layer) can be used as the uniform light layer outside the photoconductive layer, and then the direct reflection paste layer (e.g. direct reflection paper), direct reflection plating or coating (e.g. mirror paint) can be used as the reflective layer outside the uniform light layer. The advantage of adopting the paste layer as the uniform light layer or reflective layer is that it is not easy to fray or fall off, while adopting plating or coating is of better uniformity and conformability, thus different schemes can be chosen according to the practical situation.

As shown in FIG. 4, in another embodiment of this invention, the upper end and lower end of the photoconductive component 5 are sealed by the upper cover 61 and the lower cover 62, respectively, and together with the front panel 63 of the housing, the photoconductive component 5, the upper cover 61 and the lower cover 62 form a closed shooting environment. In order to uniform the illumination, the upper cover and the lower cover comprises inside to outside translucent layer, uniform light layer and reflective layer. The thinner (0.5 mm˜3 mm) organic glass plate or translucent glass can be adopted as translucent layer, and the white diffuse reflection paper or white painting layer can be used as uniform light layer, as well as the direct reflection paper or mirror paint can be used as reflective layer.

Further, the photoconductive component of the invention can be designed in the structure of polygon, curved surface or spherical surface, as shown in FIG. 3 and FIG. 4, the second photoconductive plate 52 is of a curved surface structure. Accordingly, the corresponding slot 7 is positioned on the sides facing the photoconductive component 5 of the upper cover 61 and the lower cover 62, and the photoconductive component is stuck in the slot 7.

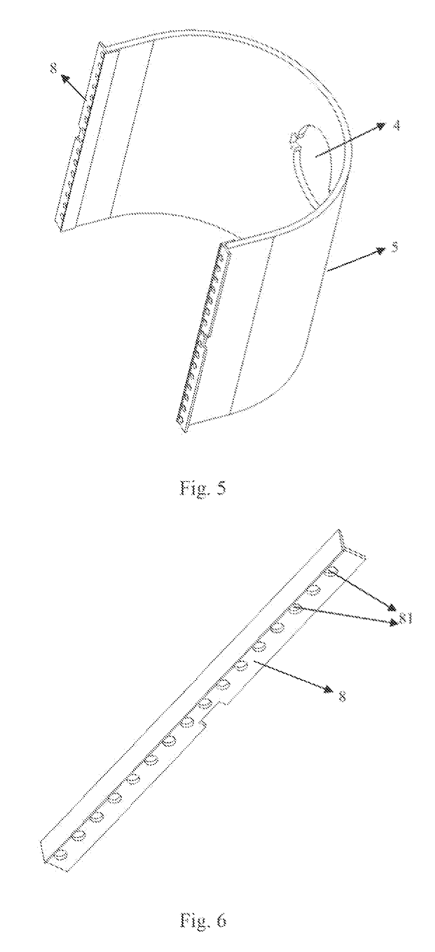

As shown in FIG. 5 and FIG. 6, in the present invention the light source adopted in the shooting device is point light source—LED light source, photoconductive component is a U type curved surface structure, and the opening of the U type curved surface structure faces the front penal of the monitoring device for inspection diagnosis in traditional Chinese medicine.

Further, the LED fixing bar 8 having the LED mounting hole 81 is positioned on the side cross section of the opening of the U type curved surface structure, and the LED is fixed in the mounting hole 81. The LED fixing bar 8 is of opaque material, and there is the reflective layer (the direct reflection paper or mirror paint can be used for the reflective layer) on the external surface of LED fixing bar 8, thus, the light loss can be avoided and all light from light source will enter the photoconductive layer of photoconductive component 5. In order to make the illumination close to the natural light, the number of LED is preferably 20 to 60, and those LEDs can be positioned in the LED fixing bar 8 on the two sides. The color rendering index (Ra) of LED in use should be no less than 85 as well as the color temperature range of LED is 4000 k˜7000 k, and image formed in this condition is of great performance on the reduction degree in color. The LED is powered with constant current with the current for each single LED 10˜30 mA, and eventually, a stable illuminating environment which is very close to the natural light is formed with the photoconductive component.

There are two advantages with LED fixing bar 8 in the invention: one is to facilitate the complete installation and positioning of numeric LEDs so that to prevent light leakage; the other is that the opening of the U type curved surface structure faces the front penal of the monitoring device for inspection diagnosis in traditional Chinese medicine so that it is easy to disassemble and repair.

Another light source that can be adopted in the monitoring device for inspection diagnosis in traditional Chinese medicine of this invention is strip source, e.g. Cold Cathode Fluorescent Lamp shortened CCFL. The fluorescent lamp can be positioned between the adjacent photoconductive plates or on the side cross section of the photoconductive plate (e.g. the upper and the lower cross sections or the left and the right cross sections), and a shading bar should also be positioned on the surface of the photoconductive plate where the fluorescent lamp located, thus either the outleakage of the direct rays from the light source or the irradiation to the internal illumination environment can be avoided.

Other light sources can also be used in the invention, e.g. electroluminescence (EL) or Organic Light-Emitting Diode (OLED) which is also called Organic Electroluminescence Display (OELD). Referring to the said LED light source and the fluorescent lamp above, different installation methods can be chosen according to the form of the light source when different light sources are adopted.

As shown in FIG. 3 to FIG. 5, in the monitoring device for inspection diagnosis in traditional Chinese medicine of the invention, a shooting hole 4 is positioned in the photoconductive component, and the shooting structure 3 is positioned outside the photoconductive component 5 with the shooting lens of the shooting structure 3 facing the shooting hole 4 in the photoconductive component 5. In most cases, the invention is applied in the hospital, and frequent disinfection is required in order to reduce cross-infection between patients, thus damage to the camera might be brought about in the disinfection process. As a result, the camera is positioned outside the internal shooting environment in the invention, and optical glass can be embedded in the shooting hole so that the internal environment can be kept sealed.

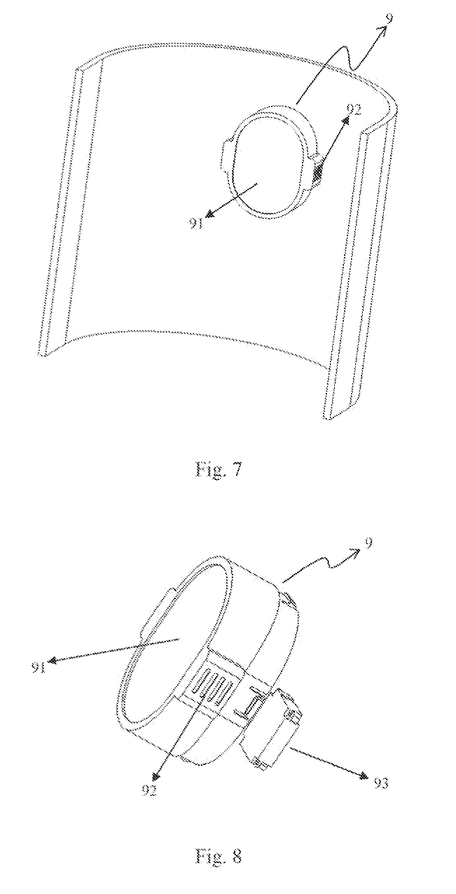

Preferably, as shown in FIG. 7, in the monitoring device for inspection diagnosis in traditional Chinese medicine of the invention, a shooting window frame 9 is positioned in the shooting hole 4, and the optical glass 91 is embedded in the shooting window frame 9, thus the shooting lens of the shooting structure 3 can be inserted into the shooting window frame 9 in order to narrow the shooting distance, whereby the size of the monitoring device for inspection diagnosis in traditional Chinese medicine can be reduced.

Further, as shown in FIG. 8, in the monitoring device for inspection diagnosis in traditional Chinese medicine of the invention, at least one air outlet 92 which is connected with the fan 93 is positioned in the shooting window frame 9, so that the air through air outlet 92 can be extracted by the fan 93 from within the internal shooting environment, whereby the air flow is formed in the shooting environment to keep the air in the shooting environment fresh and avoid cross-infection of patients. In the invention, the fan 93, which can be flexibly detached from the shooting window frame 9, is of a hooking structure.

In a better embodiment of this invention, the patients face the part monitored (e.g. face or tongue) to the front panel of the monitoring window of the monitoring device for inspection diagnosis in traditional Chinese medicine, then while the power turned on, a closed and stable shooting environment is formed inside the monitoring device by means of the photoconductive component with illumination close to natural light, and lens of the camera is inserted into the shooting hole in the photoconductive component so that the symptom information of the part monitored is recorded by shooting. At the same time, by means of the running the fan, the air flow can be formed in the shooting environment in order to comfort the monitoring environment for the patients. After the monitoring, the disinfection to the internal environment can be conducted. Inside the photoconductive component there is the photoconductive plate with the advantages of waterproof and corrosion-resistant that can be directly cleaned with alcohol. Furthermore, contamination and damage to the lens can be effectively prevented by the optical glass in shooting window frame in the disinfection process.

1. A monitoring device for inspection diagnosis in traditional Chinese medicine, comprising: a housing, a light source and a shooting structure, the light source and shooting structure are positioned in the housing, characterized in that a photoconductive component is positioned in the housing, the photoconductive component comprising at least one photoconductive layer, the light source is positioned inside the photoconductive layer or on a side cross section of the photoconductive layer, wherein a diffuse reflection through the photoconductive component of the said light source forms an area light source.

2. The monitoring device for inspection diagnosis in traditional Chinese medicine according to claim 1 characterized in that the photoconductive layer is composed by a photoconductive plate.

3. The monitoring device for inspection diagnosis in traditional Chinese medicine according to claim 1 characterized in that the photoconductive component further includes a uniform light layer outside the photoconductive layer.

4. The monitoring device for inspection diagnosis in traditional Chinese medicine according to claim 3 characterized in that the uniform light layer is a white paste layer, a white plating or a white coating.

5. The monitoring device for inspection diagnosis in traditional Chinese medicine according to claim 3 characterized in that the photoconductive component further includes a reflective layer outside the uniform light layer.

6. The monitoring device for inspection diagnosis in traditional Chinese medicine according to claim 5 characterized in that the reflective layer is a direct reflection paste layer, a direct reflection plating or a direct reflection coating.

7. The monitoring device for inspection diagnosis in traditional Chinese medicine according to claim 1 characterized in that an upper end and a lower end of the photoconductive component are sealed by an upper cover and a lower cover respectively, and the photoconductive component, the upper cover and the lower cover form a closed shooting environment.

8. The monitoring device for inspection diagnosis in traditional Chinese medicine according to claim 7 characterized in that a slot corresponding to the photoconductive component is positioned on sides of the upper cover and the lower cover facing the photoconductive component, and the photoconductive component is disposed in the slot.

9. The monitoring device for inspection diagnosis in traditional Chinese medicine according to claim 1 characterized in that the photoconductive component is in the shape of a polygon, a curved surface or a spherical surface.

10. The monitoring device for inspection diagnosis in traditional Chinese medicine according to claim 1 characterized in that the light source is a point light source.

11. The monitoring device for inspection diagnosis in traditional Chinese medicine according to claim 10 characterized in that the light source is a plurality of LEDs, numbering between about 20 and 60.

12. The monitoring device for inspection diagnosis in traditional Chinese medicine according to claim 11 characterized in that a color rendering index (Ra) of the LEDs in use is no less than 85, and the color temperature range of the LEDs is about 4000 k to 7000 k.

13. The monitoring device for inspection diagnosis in traditional Chinese medicine according to claim 11 characterized in that the LEDs are powered with a constant current with the current for each LED being about 10 to 30 mA.

14. The monitoring device for inspection diagnosis in traditional Chinese medicine according to claim 11 characterized in that the photoconductive component is a U type curved surface structure, and an opening of the U type curved surface structure faces a front panel of the monitoring device, the LEDs being positioned on a side cross section of the opening of the U type curved surface structure.

15. The monitoring device for inspection diagnosis in traditional Chinese medicine according to claim 14 characterized in that an LED fixing bar with LED mounting holes is positioned on the side cross section of the opening of the U type_curved surface structure, the LEDs being fixed in the mounting holes.

16. The monitoring device for inspection diagnosis in traditional Chinese medicine according to claim 15 characterized in that the LED fixing bar is an opaque material.

17. The monitoring device for inspection diagnosis in traditional Chinese medicine according to claim 15 characterized in that a reflective layer is disposed on an external surface of LED fixing bar.

18. The monitoring device for inspection diagnosis in traditional Chinese medicine according to claim 1 characterized in that the light source is a strip source.

19. The monitoring device for inspection diagnosis in traditional Chinese medicine according to claim 18 characterized in that a color rendering index (Ra) of the strip source is no less than 85.

20. The monitoring device for inspection diagnosis in traditional Chinese medicine according to claim 18 characterized in that a shading bar is positioned on a surface of the photoconductive plate where a fluorescent lamp is located.

21. The monitoring device for inspection diagnosis in traditional Chinese medicine according 1 characterized in that a shooting hole is positioned in the photoconductive component, the shooting structure being positioned outside the photoconductive component with the shooting lens of the shooting structure facing the shooting hole in the photoconductive component.

22. The monitoring device for inspection diagnosis in traditional Chinese medicine according to claim 21 characterized in that optical glass is embedded in the shooting hole.

23. The monitoring device for inspection diagnosis in traditional Chinese medicine according to claim 21 characterized in that a shooting window frame is positioned in the shooting hole, and the optical glass is embedded in the shooting window frame.

24. The monitoring device for inspection diagnosis in traditional Chinese medicine according to claim 23 characterized in that at least one air outlet is in communication with a fan positioned in the shooting window frame.
